(19) United States
(12) Patent Application Publication
(10) Pub. No.: US 2015/0086985 A1
(43) Pub. Date: Mar. 26, 2015

(54) CELLULAR UPTAKE CONTROL SYSTEMS

(71) Applicants: David A. Giljohann, Chicago, IL (US); Richard Kang, Evanston, IL (US)

(72) Inventors: David A. Giljohann, Chicago, IL (US); Richard Kang, Evanston, IL (US)

(73) Assignee: AuraSense, LLC, Skokie, IL (US)

(21) Appl. No.: 14/344,268

(22) PCT Filed: Sep. 11, 2012
(86) PCT No.: PCT/US2012/054636

§ 371 Date: Oct. 21, 2014

Related U.S. Application Data
(60) Provisional application No. 61/533,238, filed on Sep. 11, 2011.

(51) Int. Cl. C12Q 1/68; C07F 1/00

(52) U.S. Cl. CPC C12Q 1/6825; C07F 1/005
USPC 435/6.11; 435/7.1; 536/23.1; 435/366

(57) ABSTRACT

Aspects of the invention relate to novel methods and compositions for assessing the level of cellular uptake of a nanoparticle construct, assessing the level of target binding of a nanoparticle construct and assessing the levels of RNAs and proteins in a given cell.

### RELATED APPLICATIONS

This application claims the benefit under 35 U.S.C. §119(e) of U.S. Provisional Application Ser. No. 61/533,238, entitled “INTRACELLULAR QUANTITATIVE DETECTION OF MRNA TARGETS,” filed on Sep. 11, 2011, the disclosure of which is incorporated by reference herein in its entirety.

### FIELD OF INVENTION

The invention relates to methods and compositions for monitoring the cellular uptake of a particle.

### BACKGROUND OF INVENTION

Delivery of constructs, such as nanoparticle constructs, to cells is a central aspect of many therapeutic and diagnostic approaches. For example, nanoparticle constructs can be used to deliver drugs such as chemotherapeutic agents or binding moieties such as oligonucleotides or antibodies that will bind to and regulate expression of a biological molecule in a cell, and that can be used to assess the levels of molecules such as RNAs and proteins in cells.

For many applications involving delivery of a nanoparticle construct, it is relevant to determine the level of cellular uptake of the construct, such as for determining effectiveness of delivery and/or assessing recommended dosage of a construct. The same nanoparticle construct can enter different types of cells at different rates. Consequently, the same construct may have different effects on different types of cells. It is difficult to accurately determine the level of cellular uptake of a construct and to accurately compare cellular uptake between different cell types. Generally, in order to determine whether the different effects of a construct between cells are the result of differences in cellular uptake and to normalize for the uptake, an analytical assay is necessary. For metal particles, an example of such an assay is inductively coupled plasma mass spectrometry (ICP-MS). Using ICP-MS, one can determine the amount of metal such as gold, silver or iron present in the cells and correlate the effects as concentration of constructs present inside the cell.

### SUMMARY OF INVENTION

Described herein are novel methods and compositions for measuring cellular uptake of a nanoparticle construct. Aspects of the invention relate to a method for detecting cellular uptake of a nanoparticle construct, comprising: providing a nanoparticle construct, comprising: a nanoparticle core, a first modality comprising a binding moiety specific for a target molecule, that is attached to the nanoparticle core; and a second modality comprising an uptake control moiety, that is attached to the nanoparticle core and that includes a reference chromophore; contacting the nanoparticle construct with a cell; and detecting the level of the reference chromophore within the cell, wherein the level of the reference chromophore within the cell indicates the level of cellular uptake of the nanoparticle construct within the cell.

In certain embodiments, the nanoparticle construct comprises more than one reference chromophore. In certain embodiments, the uptake control moiety comprises a polynucleotide, a polypeptide or a polymer. In certain embodiments, one or more of the reference chromophores is a fluorophore or a quantum dot. In certain embodiments, the binding moiety and/or the uptake control moiety is linked to the nanoparticle core by a spacer.

In certain embodiments, the binding moiety is a polynucleotide or a polypeptide. In certain embodiments, wherein a polynucleotide serves as the binding moiety, the polynucleotide is RNA or DNA. In certain embodiments, the polynucleotide serving as the binding moiety is ssRNA. In certain embodiments, the polynucleotide serving as the binding moiety is dsRNA. In certain embodiments, the polynucleotide serving as the binding moiety ssDNA. In certain embodiments, wherein a polypeptide serves as the binding moiety, the polypeptide is an antibody.

In certain embodiments, the binding moiety is labeled. In certain embodiments, the label is a detectable marker that is detected when the binding moiety binds to its target molecule. In certain embodiments, the detectable marker is a chromophore.

In certain embodiments, the nanoparticle core of the nanoparticle construct is metallic. In certain embodiments, the metal is selected from the group consisting of gold, silver, platinum, aluminum, palladium, copper, cobalt, indium, nickel and mixtures thereof. In certain embodiments, the nanoparticle core comprises gold. In certain embodiments, the nanoparticle core is a lattice structure including degradable gold.

In certain embodiments, the diameter of the nanoparticle is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

In certain embodiments, the nanoparticle construct comprises multiple binding moieties. In certain embodiments, the binding moieties bind to one target molecule. In other embodiments, the binding moieties bind to multiple target molecules.

In certain embodiments, the method involves delivering a therapeutic or detection modality to a cell.

In certain embodiments, the method involves regulating expression of a target molecule.

Other aspects of the invention relate to a method for detecting binding of a nanoparticle construct to a target molecule in a cell, comprising: providing a nanoparticle construct, comprising: a nanoparticle core, a first modality comprising a binding moiety specific for a target molecule, that is attached to the nanoparticle core and that includes a first chromophore; and a second modality comprising an uptake control moiety that is attached to the nanoparticle core and that includes a second chromophore; contacting the nanoparticle construct with a cell; and detecting the levels of the first and second chromophores within the cell, wherein the level of the first chromophore relative to the level of the second chromophore, is indicative of the level of binding of the nanoparticle construct to a target molecule in a cell.

In certain embodiments, the nanoparticle construct comprises more than one reference chromophore. In certain embodiments, the uptake control moiety comprises a polynucleotide, a polypeptide or a polymer. In certain embodiments, one or more of the reference chromophores is a fluorophore or a quantum dot. In certain embodiments, the binding moiety and/or the uptake control moiety is linked to the nanoparticle core by a spacer.

In certain embodiments, the binding moiety is a polynucleotide or a polypeptide. In certain embodiments, wherein a polynucleotide serves as the binding moiety, the polynucleotide is RNA or DNA. In certain embodiments, the polynucleotide serving as the binding moiety is ssRNA. In certain embodiments, the polynucleotide serving as the binding moiety is dsRNA. In certain embodiments, the polynucleotide serving as the binding moiety ssDNA. In certain embodiments, wherein a polypeptide serves as the binding moiety, the polypeptide is an antibody.

In certain embodiments of the method for detecting binding of a nanoparticle construct in a target molecule in a cell, the first chromophore is detected when the binding moiety binds to its target molecule.

In certain embodiments, the nanoparticle core is metallic. In certain embodiments, the metal is selected from the group consisting of gold, silver, platinum, aluminum, palladium, copper, cobalt, indium, nickel and mixtures thereof. In certain embodiments, the nanoparticle core comprises gold. In certain embodiments, the nanoparticle core is a lattice structure including degradable gold.

In certain embodiments, the diameter of the nanoparticle is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

In certain embodiments, the nanoparticle comprises multiple binding moieties. In certain embodiments the binding moieties bind to one target molecule. In other embodiments, the binding moieties bind to multiple target molecules.

In certain embodiments, the method involves delivering a therapeutic or detection modality to a cell.

In certain embodiments, the method involves regulating expression of a target molecule.

Other aspects of the invention relate to a nanoparticle construct comprising: a nanoparticle core; a first modality comprising a binding moiety specific for a target molecule, that is attached to the nanoparticle core; and a second modality comprising an uptake control moiety that is attached to the nanoparticle core and that includes a reference chromophore.

In certain embodiments, this nanoparticle construct comprises more than one chromophore. In certain embodiments, the one or more reference chromophores is a fluorophore or a quantum dot.

In certain embodiments, the uptake control moiety is attached to the nanoparticle core through a polynucleotide, a polypeptide or a polymer.

In certain embodiments, the binding moiety and/or the uptake control moiety is linked to the nanoparticle core by a spacer. In certain embodiments, the binding moiety is a polynucleotide or a polypeptide. In certain embodiments, wherein a polynucleotide serves as the binding moiety, the polynucleotide is RNA or DNA. In certain embodiments, the polynucleotide serving as the binding moiety is ssRNA. In certain embodiments, the polynucleotide serving as the binding moiety is dsRNA. In certain embodiments, the polynucleotide serving as the binding moiety ssDNA. In certain embodiments, wherein a polypeptide serves as the binding moiety, the polypeptide is an antibody.

In certain embodiments, the binding moiety is labeled. In certain embodiments, this label is a detectable marker that is detected when the binding moiety binds to its target. In certain embodiments, the detectable marker serving as a label is a chromophore.

In certain embodiments, the nanoparticle core is metallic. In certain embodiments, the metal is selected from the group consisting of gold, silver, platinum, aluminum, palladium, copper, cobalt, indium, nickel and mixtures thereof. In certain embodiments, the nanoparticle core comprises gold. In certain embodiments, the nanoparticle core is a lattice structure including degradable gold.

In certain embodiments, the diameter of the nanoparticle is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

In certain embodiments, the nanoparticle construct comprises multiple binding moieties. In certain embodiments of this nanoparticle construct, the binding moieties bind to one target molecule. In other embodiments, the binding moieties bind to multiple target molecules.

Further aspects of the invention relate to a method for delivering a therapeutic or detection modality to a cell comprising delivering the nanoparticle construct of the composition described to the cell.

Further aspects of the invention relate to a kit comprising: a nanoparticle; a modality comprising a binding moiety specific for a target molecule; and a modality comprising a reference chromophore. In certain embodiments, the kit further comprises instructions for use.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a non-limiting example of a nanoparticle construct containing a double-stranded therapeutic and/or detection modality.

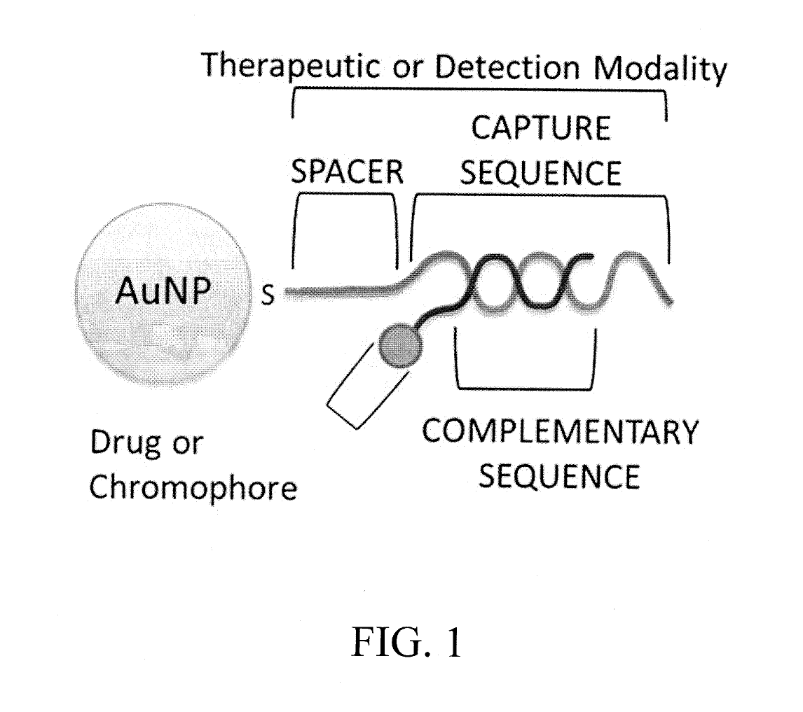

FIG. 2 shows a non-limiting example of a nanoparticle construct containing a double-stranded therapeutic and/or detection modality and an uptake control oligonucleotide bearing a reference chromophore.

FIG. 3 demonstrates that uptake control oligonucleotides with a Cy3 chromophore load at consistent levels in different batch preparations.

FIG. 4 demonstrates that multiple uptake control oligonucleotides can be incorporated into the same nanoparticle construct to add robustness to the uptake normalization signal. For example, the chromophores Cy5 and Cy3 are shown in this non-limiting example. Both chromophores can be consistently loaded on the nanoparticle to make nanoparticle constructs that are identical across different preparations.

FIG. 5 demonstrates cellular uptake of different formulations of nanoparticle constructs containing Cy5- and Cy3-labeled oligonucleotides.

### DETAILED DESCRIPTION

Aspects of the invention relate to novel methods and compositions for assessing the level of cellular uptake of a nanoparticle construct, assessing the level of target binding of a nanoparticle construct and assessing the levels of RNAs and proteins in a given cell. The invention is based, at least in part, on the development of an uptake control moiety that can be attached to a nanoparticle construct and that contains a chromophore. Detection of a signal such as fluorescence from the chromophore of the uptake control moiety can be used to assess the level of cellular uptake of a nanoparticle construct. In some aspects, if the nanoparticle construct contains multiple chromophores, then assessment of the level of cellular uptake and/or target binding of the nanoparticle construct can involve a comparison of the levels of signals, such as fluorescent signals, of more than one chromophore.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to nanoparticle constructs. A nanoparticle construct refers to a nanoparticle core that is attached to one or more other modalities. As used herein, a nanoparticle is a particle having an average diameter on the order of nanometers (i.e., between about 1 nm and about 1 micrometer. For example, in some instances, the diameter of the nanoparticle is from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, about 1 nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

As used herein, a nanoparticle core refers to the nanoparticle component of a nanoparticle construct, without any attached modalities. In some instances, the nanoparticle core is metallic. It should be appreciated that the nanoparticle core can comprise any metal. Several non-limiting examples of metals include gold, silver, platinum, aluminum, palladium, copper, cobalt, indium, nickel and mixtures thereof. In some embodiments, the nanoparticle core comprises gold. For example, the nanoparticle core can be a lattice structure including degradable gold. Nanoparticles can also comprise semiconductor and magnetic materials.

Non-limiting examples of nanoparticles compatible with aspects of the invention are described in and incorporated by reference from: U.S. Pat. No. 7,238,472, US Patent Publication No. 2003/0147966, US Patent Publication No. 2008/0306016, US Patent Publication No. 2009/0209629, US Patent Publication No. 2010/0136682, US Patent Publication No. 2010/0184844, US Patent Publication No. 2010/0294952, US Patent Publication No. 2010/0129808, US Patent Publication No. 2010/0233270, US Patent Publication No. 2011/0111974, PCT Publication No. WO 2002/096262, PCT Publication No. WO 2003/08539, PCT Publication No. WO 2006/138145, PCT Publication No. WO 2008/127789, PCT Publication No. WO 2008/098248, PCT Publication No. WO 2011/079290, PCT Publication No. WO 2011/053940, PCT Publication No. WO 2011/017690 and PCT Publication No. WO 2011/017456. Nanoparticles associated with the invention can be synthesized according to any means known in the art or can be obtained commercially. For example, several non-limiting examples of commercial suppliers of nanoparticles include: Ted Pella, Inc., Redding, Calif., Nanoprobes, Inc., Yaphank, N.Y., Vacuum Metallurgical Co,. Ltd., Chiba, Japan and Vector Laboratories, Inc., Burlington, Calif.

### Binding Moieties

The nanoparticle core of a nanoparticle construct can be attached to one or more modalities. In some instances, the nanoparticle core is attached to a modality that comprises a binding moiety that has specificity for binding to a target molecule in a cell. A binding moiety can be any moiety that has the capability of binding to a specific target molecule on a cell. For example, a binding moiety can be a polynucleotide, a polypeptide or a small molecule. In some aspects, a binding moiety is a polynucleotide, such as RNA or DNA. For example, a binding moiety that comprises RNA can be an ssRNA, a dsRNA or any other RNA molecule that has specificity for binding to a target molecule in a cell or can be a ssDNA or any other DNA molecule that has specificity for binding to a target molecule in a cell. In other instances, a binding moiety is a polypeptide, such as an antibody, that has specificity for binding to a target molecule in a cell.

In some aspects, the binding moiety is not labeled. In other aspects, the binding moiety is labeled, such as with a detectable marker. For example, the binding moiety can be labeled with a chromophore, such as a fluorophore. In some aspects, the labeling of a binding moiety allows for detection of a binding event in a cell because the signal becomes detectable upon the binding moiety binding to its target molecule in the cell. For example, the nanoparticle construct can be a nanoflare, which can be used to detect targets in a cell in a quantitative fashion. A nanoflare refers to a three dimensional organization of oligonucleotides, as described further in US Patent Publication No. 2010/0129808 and PCT Publication No. WO/2008/098248 (PCT Application Serial No. PCT/US2008/053603), each of which is incorporated by reference herein in its entirety. The arrangement of oligonucleotides in a nanoflare provides for cellular uptake, resistance to nuclease degradation, and the release of a detectable signal in response to binding to a target. Importantly, the signal of the nanoflare remains quenched due to the proximity between the signaling agent and a quencher. Upon hybridization or binding to a target molecule of interest in a cell, the signal is released. Nanoparticle constructs can comprise multiple binding moieties that can bind to one or more target molecules.

The binding moiety can be a therapeutic or detection moiety. As used herein a therapeutic moiety is a moiety that is administered to a cell for therapeutic purposes, such as for delivery of a drug or biologic, including a polynucleotide such as DNA or RNA, including antisense DNA, siRNA, miRNA, antisense RNA, oligonucleotides such as triplex forming oligonucleotides, aptamers and antibodies. A detection moiety is a moiety that is administered to a cell to detect an RNA or protein, such as for diagnostic purposes.

### Uptake Control Moiety

Nanoparticle constructs associated with the invention are attached to a modality comprising an uptake control moiety. As used herein, an uptake control moiety refers to a reference chromophore and an attachment means for linking the chromophore to the nanoparticle core. For example, an uptake control moiety can comprise a polynucleotide, a polypeptide or a polymer for attaching the reference chromophore to the nanoparticle core. In nanoparticle constructs wherein the uptake control moiety is a polynucleotide, in some instances, the polynucleotide does not have a target in the transcriptome of a cell. In other instances, the polynucleotide does have a target in the transcriptome of a cell. For example, the polynucleotide can bind to a housekeeping gene within a cell, such as GAPDH or β-Actin.

The uptake control moiety contains a reference chromophore which is used to determine the relative uptake of a nanoparticle construct in a cell. In some instances, the uptake control moiety can alter the cellular uptake of nanoparticle constructs. However, its presence on every construct may ensure that the uptake is uniform across all the cells in a sample. The uptake control moiety can be synthesized such that it has no effect on cell health. In other embodiments, the uptake control moiety can be synthesized so that it has a therapeutic effect. The reference chromophore is orthogonal to other chromophores that may be used as part of the binding moiety.

An uptake control moiety can contain any chromophore suitable for detection. In addition, multiple chromophores can be attached to each nanoparticle core which can improve robustness of uptake comparison. If the chromophores are present on different uptake control moieties, then the presence of each individual uptake control moiety and its contribution to the cellular uptake can be determined.

Measuring Cellular Uptake and/or Target Binding of a Nanoparticle Construct

Aspects of the invention relate to the synthesis of nanoparticle constructs that comprise uptake control moieties and the use of uptake control moieties to measure cellular uptake of a nanoparticle construct. For example, a nanoparticle construct associated with the invention can have one or more binding moieties attached to the nanoparticle core and one or more uptake control moieties comprising reference chromophores attached to the nanoparticle core. The binding moieties may or may not be labeled. If the binding moieties are not labeled, then detection of the reference chromophore(s) provides a means for assessing the level of the nanoparticle construct, including the binding moiety, taken up by a cell. Accordingly, in embodiments where it is not preferable to label a binding moiety, such as if a label may interfere with the therapeutic or diagnostic purposes of the binding moiety, then an uptake control moiety can be used to assess the level of cellular delivery of the binding moiety since both the binding moiety and the uptake control moiety are attached to the same nanoparticle construct.

Other aspects of the invention relate to using an uptake control moiety to measure the binding of a nanoparticle construct to a target molecule in a cell. In some aspects, the binding moiety is labeled, such as in the example of a nanoflare, discussed above, wherein the binding moiety is an oligonucleotide containing a chromophore that is detected when the oligonucleotide binds to its target molecule. A labeled binding moiety can also be a polypeptide, such as an antibody, or a small molecule. Once a nanoparticle construct, such as a nanoflare, is synthesized, characterized, and transfected into a cell, the signal from the reference chromophore on the uptake control moiety can be subtracted or divided from the signal from the nanoflare to delineate the portion of the signal, such as a fluorescent signal, emanating from the nanoflare that is due to target binding, thereby allowing for qualitative and quantitative comparisons of target binding and/or gene expression levels across cell types.

Methods described herein, involving detection of one or more chromophores on a nanoparticle construct, are less intrusive approaches for assessing cellular uptake of a nanoparticle construct, measuring target binding by a nanoparticle construct and measuring levels of a target molecule, such as an RNA or protein in a cell. In contrast to previous methods such as RTPCR for measuring RNA levels in cells and cell lysates or ICP-MS for detecting the amount of metal present in a cell, methods described herein utilize techniques such as flow cytometry and image-based analysis. Unlike RTPCR, which requires the lysis of a population of cells to measure its average gene expression, using nanoparticle constructs described herein, RNA levels can be measured in individual live cells. In addition, differences in RNA levels can be used for fluorescence activated cell sorting (FACS) in a similar but much broader manner to existing protein based population analysis and sorting.

FACS is commonly used to separate cells in populations by flow cytometry. One common sorting method is to interrogate populations based on cell surface proteins. Typically, membrane-bound proteins on cells are labeled with fluorescent dye-conjugated antibodies, washed and then analyzed by FACS. Cells bearing the antigen of interest are highly fluorescent when compared to cells without the membrane proteins. Those fluorescent cells are then mechanically separated from the low fluorescence signal population.

FACS based on the RNA content of live cells is challenging but represents an extremely powerful technique since FACS is generally based on a limited number of cell surface proteins and cell permeable dye-based assays. By contrast, nanoparticle constructs can interrogate the entire transcriptome. Nanoparticle constructs described herein allow for unprecedented levels of live cell genotypic characterization and cell sorting, resulting in highly useful tools for researchers and clinicians.

Methods described herein may be carried out in vitro, ex vivo, or in vivo, including, for example, mammalian cells in culture, such as a human cell in culture.

The target cells (e.g., mammalian cell) may be contacted in the presence of a delivery reagent, such as a lipid (e.g., a cationic lipid) or a lipo some.

Another aspect of the invention provides a method for regulating the expression of a target gene in a mammalian cell, comprising contacting the mammalian cell with a nanoparticle construct.

The Uptake Control Moiety Allows for Quantification of Biologically Relevant Signals within Cells and between Cells

A limitation of previous nanoparticle constructs, such as nanoflares, was their inability to accurately quantify intracellular RNA levels due to background fluorescence within cells. Herein, relative RNA quantification with nanoparticle constructs can be achieved by modifying nanoparticle constructs to include an uptake control moiety that contains a chromophore that is distinct from the chromophore attached to the binding moiety of the nanoparticle construct. The signal of the reference chromophore is subtracted or divided from the signal of the chromophore attached to the binding moiety allowing for determination of the binding-specific signal of the binding moiety. In some instances, the uptake control moiety comprises an oligonucleotide that binds to a target gene such as GAPDH or β-Actin, while the binding moiety binds to a gene of interest, such that the comparison of fluorescent signals gives relative gene levels. In other instances, the uptake control moiety does not bind to a gene within a cell. The reference chromophore allows for normalization of a target binding signal by allowing assessment of the level of cellular uptake of the nanoparticle construct and by allowing for subtraction of background fluorescence.

Another limitation with the use of labeled nanoparticle constructs, such as nanoflares, has been the inability to accurately compare gene expression levels across cell types. Nanoparticle constructs, such as nanoflares, are taken up and degraded by different cell types and within individual cells in a population at different rates. While the fluorescence detected from a nanoflare should, in principle, reflect a biologically meaningful binding event in a cell, greater cellular uptake and degradation of the nanoflare increases fluorescence signal within the cell, causing a high level of background which becomes indistinguishable from a binding event signal. In order to deconvolute the contributions of uptake, degradation and target binding events from each other, methods and compositions described herein involve normalizing the signal detected by the binding moiety by subtracting out the fluorescence detected from the reference chromophore. The synthesis of nanoparticle constructs that comprise uptake control moieties, which have similar uptake and stability to the RNA targeted nanoflares, is described herein.

### Labeling of Uptake Control Moieties and Binding Moieties

Uptake control moieties associated with the invention comprise chromophores to permit their detection in cells. A cell can have one or more uptake control moieties and one or more reference chromophores. Non-limiting examples of chromophores include fluorophores and quantum dots. Oligonucleotides can be labeled with chromophores according to any means known in the art. Labeling moieties can include fluorescent, colorimetric, radioactive, quantum dots, NIR active, magnetic, catalytic, enzymatic, protein, mass tags and chemiluminescent agents.

In some instances, the chromophore is a fluorophore. Several non-limiting examples of fluorophores include: 1,8-ANS (1-Anilinonaphthalene-8-sulfonic acid), 1-Anilinonaphthalene-8-sulfonic acid (1,8-ANS), 5-(and-6)-Carboxy-2′,7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyhrodamine 6G pH 7.0, 6-Carboxyrhodamine 6G, hydrochloride, 6-HEX, SE pH 9.0, 6-TET, /SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl)sulfonamide, DDAO pH 9.0, Di-8 ANEPPS, Di-8-ANEPPS-lipid, Di, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Co, Fura-2 Ca2+, Fura-2, high Cu” Fura-2, no Cu, GFP (S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Nissl stain-RNA, Nile Blue, EtOH, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PicoGreen dsDNA quantitation reagent, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, EtOH, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

It should be appreciated that other types of detectable markers and labels are compatible with aspects of the invention as described further in, and incorporated by reference from, US Patent Publication No. 2010/0129808.

Quenching moieties compatible with aspects of the invention include Dabcyl, Malachite green, QSY 7, QSY 9, QSY 21, QSY 35, Iowa Black and Black Hole Quenchers, proteins and peptides. The nanoparticle core can also serve as a quencher in the absence of or in addition to the presence of other quenching moieties.

Attachment of modalities to nanoparticles

Modalities associated with the invention, including uptake control moieties and binding moieties, such as polynucleotides, polypeptides and small molecules, can be attached to nanoparticle cores by any means known in the art. Methods for attaching oligonucleotides to nanoparticles are described in detail in and incorporated by reference from US Patent Publication No. 2010/0129808.

A nanoparticle can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, polynucleotides are attached to nanoparticles by terminating the polynucleotide with a 5′ or 3′ thionucleoside. In other instances, an aging process is used to attach polynucleotides to nanoparticles as described in and incorporated by reference from U.S. Pat. Nos. 6,361,944, 6,506, 569, 6,767,702 and 6,750,016 and PCT Publication Nos. WO 1998/004740, WO 2001/000876, WO 2001/051665 and WO 2001/073123.

In some instances, the uptake control moiety and/or the binding moiety are covalently attached to the nanoparticle core, such as through a gold-thiol linkage. A spacer sequence can be included between the attachment site and the uptake control moiety and/or the binding moiety. In some embodiments, a spacer sequence comprises or consists of an oligonucleotide, a peptide, a polymer or an oligoethylene.

Nanoparticle constructs can be designed with multiple chemistries. For example, a DTPA (dithiol phosphoramidite) linkage can be used. The DTPA resists intracellular release of flares by thiols and can serve to increase signal to noise ratio.

### Polynucleotides

The terms “nucleic acid,” “polynucleotide” and “oligonucleotide” are used interchangeably herein to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g., genomic or cDNA), but are preferably synthetic (e.g. produced by nucleic acid synthesis). In some embodiments, the polynucleotide is a DNA, RNA or LNA molecule.

A polynucleotide attached to a nanoparticle core can be single stranded or double stranded. A double stranded polynucleotide is also referred to herein as a duplex. Double-stranded oligonucleotides of the invention can comprise two separate complementary nucleic acid strands.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded polynucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded polynucleotide of the invention contains at least or up to 1, 2, 3, 4, 5, 6, 7, 8, 9, 10, 11, 12, 13, 14, or 15 mismatches.

Polynucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH₂, NHR, NR₂,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In some embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In some embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulffiydryl, alkylthio, arylthio, thiocarboxylate, sulfates, alkylsulfmyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “hydrophobic modifications” refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; naphthyl,

The term “heteroatom” includes atoms of any element other than carbon or hydrogen. In some embodiments, preferred heteroatoms are nitrogen, oxygen, sulfur and phosphorus. The term “hydroxy” or “hydroxyl” includes groups with an —OH or —O⁻ (with an appropriate counterion). The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃0(CR′R″)₀₋₃H, (CR′R″)₀₋₃S(O)₀₋₂R′, (CR′R″)₀₋₃0(CR′R″)₀₋₃H, (CR′R″)₀₋₃COR′, (CR′R″)₀₋₃CO₂R′, or (CR′R″)₀₋₃0R′ groups; wherein each R′ and R″ are each independently hydrogen, a C₁-0₅ alkyl, C₂-0₅ alkenyl, C₂-0₅ alkynyl, or aryl group, or R′ and R″ taken together are a benzylidene group or a —(CH₂)₂0(CH₂)₂-group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, the nucleomonomers of a polynucleotide of the invention are RNA nucleotides, including modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO²)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some aspects, polynucleotides can comprise both DNA and RNA. In some aspects, at least a portion of the contiguous polynucleotides are linked by a substitute linkage, e.g., a phosphorothioate linkage. The presence of substitute linkages can improve pharmacokinetics due to their higher affinity for serum proteins.

In some aspects, antisense (guide) sequences can include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Advantages conferred by morpholino oligonucleotides include: resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides also offer low toxicity at high doses. Morpholino oligonucleotides are further discussed in Antisense & Nucl. Acid Drug Dev. 1997. 7:187. Modifications of polynucleotides are discussed further in and incorporated by reference from US Patent Publication No. 2010/0129808.

### Polypeptides

In some instances, the binding moiety is a polypeptide. As used herein, the terms “protein” and “polypeptide” are used interchangeably and thus the term polypeptide may be used to refer to a full-length polypeptide and may also be used to refer to a fragment of a full-length polypeptide. The polypeptide can be a synthetic polypeptide. As used herein, the term “synthetic” means artificially prepared. A synthetic polypeptide is a polypeptide that is synthesized and is not a naturally produced polypeptide molecule (e.g., not produced in an animal or organism). It will be understood that the sequence of a natural polypeptide (e.g., an endogenous polypeptide) may be identical to the sequence of a synthetic polypeptide, but the latter will have been prepared using at least one synthetic step.

The polypeptide can be an isolated antibody or antigen-binding fragment thereof that bind specifically to a polypeptide in a cell. In certain embodiments the antibody or antigen-binding fragment thereof is attached to a detectable label.

As used herein, the term “antibody” refers to a protein that may include at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding fragment” of an antibody as used herein, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546) which consists of a VH domain or the variable domain of a heavy-chain antibody, such as a camelid heavy-chain antibody (e.g. VHH); (vi) an isolated complementarity determining region (CDR); and (vii) polypeptide constructs comprising the antigen-binding fragments of (i)-(vi). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art, such as expression of recombinant nucleic acids. The fragments are screened for utility in the same manner as are intact antibodies.

Isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g., IgM or IgG1) that is encoded by heavy chain constant region genes. Antibodies of the invention can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)2 fragment, and a Fv fragment.

Antibodies can be polyclonal, monoclonal, or a mixture of polyclonal and monoclonal antibodies. Antibodies of the invention can be produced by methods disclosed herein or by a variety of techniques known in the art. The term “monoclonal antibody,” as used herein, refers to a preparation of antibody molecules of single molecular composition. A monoclonal antibody displays a single binding specificity and affinity for a particular epitope. A monoclonal antibody displays a single binding specificity and affinity for a particular epitope. The term “polyclonal antibody” refers to a preparation of antibody molecules that comprises a mixture of antibodies active that specifically bind a specific antigen.

In other embodiments, antibodies may be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, genetically engineered antibodies, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

### Therapeutics

Aspects of the invention relate to delivery of nanoparticle constructs to a subject for therapeutic and/or diagnostic use. The particles may be administered alone or in any appropriate pharmaceutical carrier, such as a liquid, for example saline, or a powder, for administration in vivo. They can also be co-delivered with larger carrier particles or within administration devices. The particles may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, nanoparticle constructs associated with the invention are mixed with a substance such as a lotion (for example, aquaphor) and are administered to the skin of a subject, whereby the nanoparticle constructs are delivered through the skin of the subject. It should be appreciated that any method of delivery of nanoparticles known in the art may be compatible with aspects of the invention.

For use in therapy, an effective amount of the particles can be administered to a subject by any mode that delivers the particles to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

### Kits

In another aspect, the present invention is directed to a kit including one or more of the compositions previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more nanoparticle cores, such as a nanoparticle core that comprises gold. A kit can also include one or more binding moieties, such as polynucleotides or polypeptides that have specificity for one or more target molecules in a cell. A kit can also include one or more uptake control moieties that may comprise one or more types of chromophores.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

### Design of Nanoparticle Constructs Containing an Uptake Control Oligonucleotide that Bear a Reference Chromophore

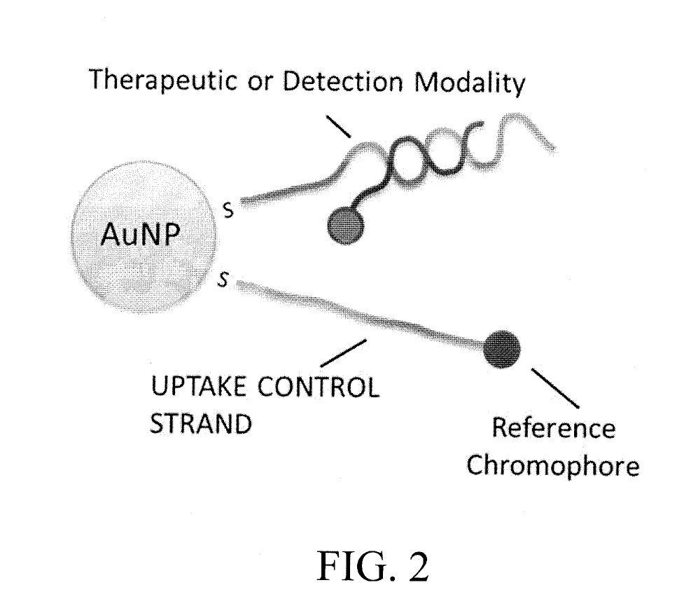

NanoFlares with an uptake control, as shown in FIG. 2, were synthesized by the following procedure. 0.7 OD/mL of 3′-DTPA capture strand oligo and 0.3 OD/mL uptake control strand with 5′-Cy3 and 3′-DTPA were mixed with 13 nm diameter gold nanoparticles (AuNPs) by vortexing and brief sonication. Next, 1.56 μL/mL 10% Tween20 was added to the oligo/AuNP mixture and vortexed. 156 μL/mL of 0.1M Phosphate buffer (pH7.4) was added to mixture and vortexed. Finally, 390 μL/mL of 2M NaCL was added to mixture and vortexed. This mixture was sonicated for 10 seconds and placed on a rotator/shaker at room temperature at low speed overnight. The following day, the mixture was washed in PBS (Ca2+/Mg2+ free) with 4 serial centrifugation steps at 15k rpm for 25 minutes. Between each centrifugation step, supernatant was aspirated and the AuNP pellet was resuspended in fresh PBS; this was sonicated such that the pellet was completely colloidal. After the final centrifugation step, the oligonucleotide functionalized AuNP was resuspended in a small volume of PBS (roughly 1/10 of the starting volume). AuNPs were quantified using UV/Vis spectrophotometry to determine absorbance at 524 nm.

Duplexing of the 5′-Cy5-labeled oligo to the functionalized AuNP was then performed by the following procedure: An excess of 50 labeled oligos/AuNP were added to the functionalized AuNP. This mixture was briefly vortexed, sonicated and then incubated for lhour at 65° C. The sample was then immediately transferred to an ice water bath for rapid cooling to 2-6° C. and incubated at 4° C. overnight. The following day, the mixture was washed in PBS with 4 serial centrifugation steps at 15k rpm for 15 minutes at RT to remove any non-duplexed oligo. Between each centrifugation step, supernatant was aspirated and the AuNP pellet resuspended in fresh PBS; this was sonicated such that the pellet was completely colloidal. After the final centrifugation step, the NanoFlare was resuspended in PBS to the desired concentration.

### Example 2

### Uptake Control Oligonucleotides with Cy3 Chromophore Load at Consistent Levels in Different Batch Preparations.

In some embodiments, it is relevant to ensure uniform loading of uptake control moieties in different batches of nanoparticles in order to be able to accurately compare both within and across batches of nanoparticles.

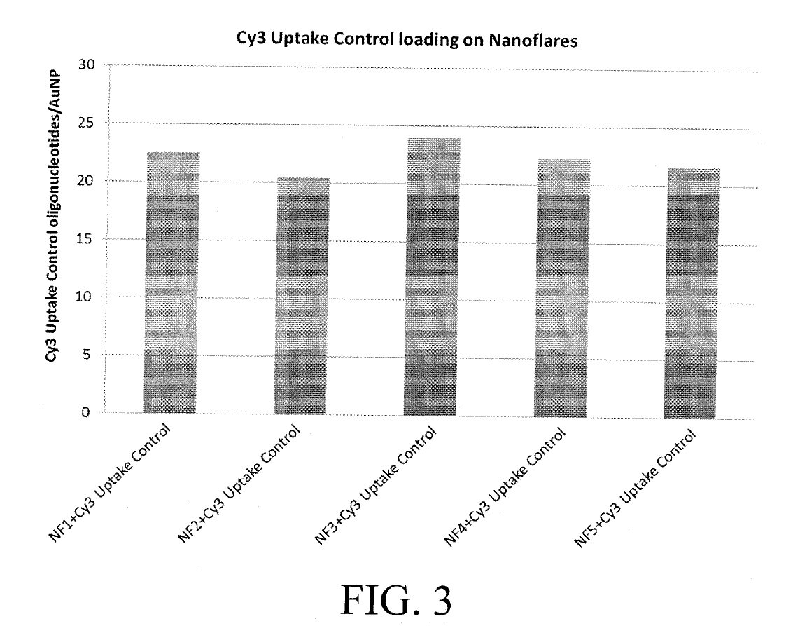

Five batches, each with unique capture sequences, were synthesized and characterized for Cy3 uptake control oligonucleotide loading per AuNP. Quantification of Cy3 uptake control oligonucleotide, as shown in FIG. 3, was performed by treating a known concentration of final functionalized nanoparticles with 125 mM KCN to oxidize the AuNP and release functionalized oligonucleotides. The loading per AuNP was calculated by measuring the fluorescence of Cy3 in these samples compared to a standard curve generated from free Cy3 uptake control oligonucleotides. All fluorescence measurements were taken using the synergy4 fluorescence plate reader.

### EXAMPLE 3

### Incorporation of Multiple Uptake Control Oligonucleotides into the Nanoparticle Construct Adds Robustness to the Uptake Normalization Signal

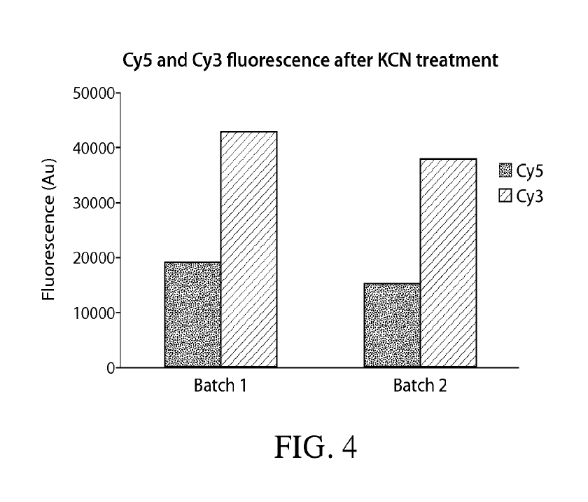

Two batches of nanoparticles were synthesized and characterized for relative numbers of Cy3- and Cy5-labeled oligonucleotides loaded on each nanoparticle. Quantification of Cy3- and Cy5-labeled oligonucleotides was performed by treating a known concentration of functionalized nanoparticles with 125 mM KCN to oxidize the AuNP and release functionalized oligonucleotides. Cy5 and Cy3 fluorescence after KCN treatment are shown FIG. 4. All fluorescence measurements were taken using the Horiba/Jobin-Yvon Fluorolog3 modular spectrofluorometer. The fluorescence from these samples was compared to a standard curve containing both Cy3- and Cy5-labeled oligonucleotides at known concentrations. This chart showed the consistency in relative fluorescence of Cy3 and Cy5 between batches (FIG. 4). Both Cy5 and Cy3 chromophores were shown to be able to be consistently loaded on the nanoparticle to make nanoparticle constructs that are identical across different preparations.

### Example 4

### Cellular Uptake of Different Formulations of Nanoparticle Constructs Containing Cy5- and Cy3-Labeled Oligonucleotides

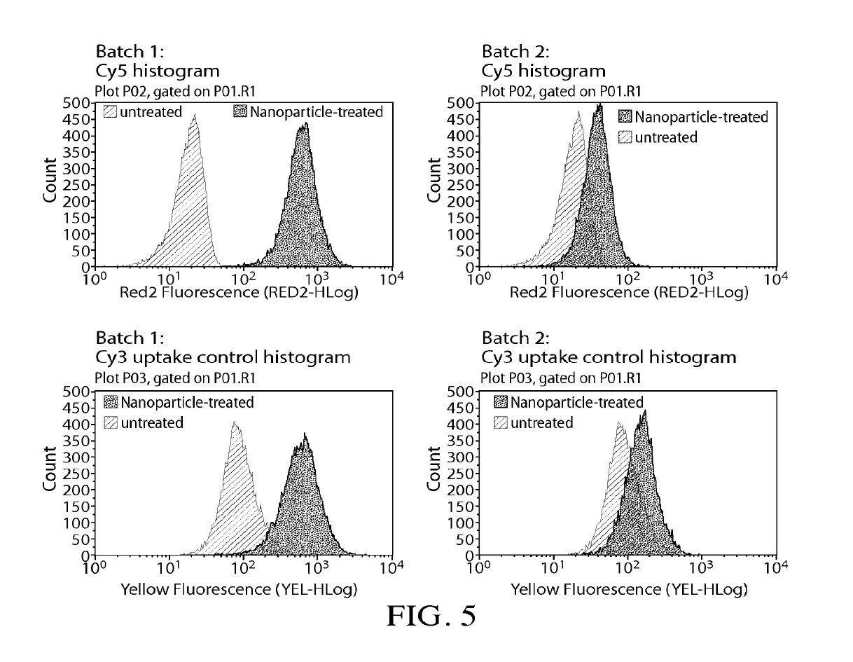

Two batches of nanoparticles with unique capture strand sequences were synthesized using the previously described method. MCF-7 cells (MEM, 10% heat inactivated FBS, 2% L-glutamine, 2% Penicillin/Streptomycin) were seeded on 96-well plates at 15,000 cells/well. After cells had fully attached to the plate surface, functionalized nanoparticles from a 2 nM stock solution (PBS) were added to each well such that the final concentration of nanoparticles was 100 μM in 100 μL cell culture medium. Plates were swirled by hand to mix the solution. Each plate was incubated for 16 hours at 37° C. Following incubation with nanoparticles, cell culture medium was aspirated and cells were washed two times with PBS. Cells were trypsinized with 30 μL of Trypsin/well until cells detached from plate. 90 μL of medium was added back to each well with vigorous pipetting to achieve a mono-dispersed cell sample. Data was analyzed for Cy5 and Cy3 emission to measure the fluorescence from each fluorophore (FIG. 5). Plates were read on a Millipore Guava 8HT flow cytometer and data was analyzed using the Incyte software suite. Relative uptake of each chromophore was determined by the fluorescence-activated cell sorting (FIG. 5). The expected chromophore level per cell type was used to normalize the uptake of the second chromophore. This determination allows measurements to be normalized across different experimental conditions, or any other variable parameter. This system can be used to monitor and determine relative delivery of various therapeutic payloads, such as antibodies, siRNA, aptamers and chemotherapeutics agents.

### Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method for detecting cellular uptake of a nanoparticle construct, comprising:
providing a nanoparticle construct, comprising:
a nanoparticle core,
a first modality comprising a binding moiety specific for a target molecule, that is attached to the nanoparticle core; and
a second modality comprising an uptake control moiety, that is attached to the nanoparticle core and that includes a reference chromophore;
contacting the nanoparticle construct with a cell; and
detecting the level of the reference chromophore within the cell, wherein the level of the reference chromophore within the cell indicates the level of cellular uptake of the nanoparticle construct within the cell.

2. The method of claim 1, wherein the nanoparticle construct comprises more than one reference chromophore.

3. The method of claim 1, wherein the uptake control moiety comprises a polynucleotide, a polypeptide or a polymer.

4. The method of claim 1, wherein one or more of the reference chromophores is a fluorophore or a quantum dot.

5. The method of claim 1, wherein the binding moiety and/or the uptake control moiety is linked to the nanoparticle core by a spacer.

6. The method of claim 1, wherein the binding moiety is a polynucleotide or a polypeptide.

7. The method of claim 6, wherein the polynucleotide is RNA or DNA.

8. The method of claim 7, wherein the polynucleotide is ssRNA.

9. The method of claim 7, wherein the polynucleotide is dsRNA.

10. The method of claim 6, wherein the polypeptide is an antibody.

11. The method of claim 1, wherein the binding moiety is labeled.

12. The method of claim 11, wherein the label is a detectable marker that is detected when the binding moiety binds to its target molecule.

13. The method of claim 12, wherein the detectable marker is a chromophore.

14. The method of claim 1, wherein the nanoparticle core is metallic.

15. The method of claim 14, wherein the metal is selected from the group consisting of gold, silver, platinum, aluminum, palladium, copper, cobalt, indium, nickel and mixtures thereof.

16. The method of claim 15, wherein the nanoparticle core comprises gold.

17. The method of claim 16, wherein the nanoparticle core is a lattice structure including degradeable gold.

18. The method of claim 1, wherein the diameter of the nanoparticle is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

19. The method of claim 1, wherein the nanoparticle construct comprises multiple binding moieties.

20. The method of claim 19, wherein the binding moieties bind to one target molecule.

21. The method of claim 19, wherein the binding moieties bind to multiple target molecules.

22. The method of claim 1, wherein the method involves delivering a therapeutic or detection modality to a cell.

23. The method of claim 1, wherein the method involves regulating expression of a target molecule.

24. The method of claim 1, wherein the method is a method for detecting binding of a nanoparticle construct to a target molecule in a cell,
wherein a first chromophore is attached to the binding moiety; and the method involves;
detecting the levels of the first and reference chromophores within the cell, wherein the level of the first chromophore relative to the level of the reference chromophore, is indicative of the level of binding of the nanoparticle construct to a target molecule in a cell.

25-44. (canceled)

45. A nanoparticle construct comprising:
a nanoparticle core;
a first modality comprising a binding moiety specific for a target molecule, that is attached to the nanoparticle core; and
a second modality comprising an uptake control moiety that is attached to the nanoparticle core and that includes a reference chromophore.

46-65. (canceled)

66. A method for delivering a therapeutic or detection modality to a cell comprising delivering the nanoparticle construct of claim 45 to the cell.

67. A method for regulating expression of a target molecule comprising delivering the nanoparticle construct of claim 45 to the cell.

68. A kit comprising:
a nanoparticle;
a modality comprising a binding moiety specific for a target molecule; and
a modality comprising a reference chromophore.

69-72. (canceled)
